(19) United States
(12) Patent Application Publication
(10) Pub. No.: US 2020/0102599 A1
(43) Pub. Date: Apr. 2, 2020

(54) ESTIMATION OF GROWTH RATE AND VIABILITY FROM GENOME COVERAGE

(71) Applicant: International Business Machines Corporation, Armonk, NY (US)

(72) Inventors: Kristen L. Beck, San Jose, CA (US); Mark Kunitomi, San Francisco, CA (US); Svetlana Lyalina, Foster City, CA (US)

(21) Appl. No.: 16/147,777

(22) Filed: Sep. 30, 2018

(51) Int. Cl. C12Q 1/6809; C12Q 1/6869; C12Q 1/689; G06F 19/22

(52) U.S. Cl. CPC C12Q 1/6809; G06F 19/22; C12Q 1/689; C12Q 1/6869

(57) ABSTRACT

A method is describe for predicting the viability and growth rate of at least one prokaryotic species of a non-cultured working sample (e.g., environmental, medical, food). The method utilizes a reference database containing time-dependent sequence data obtained from control samples of prokaryotic species at various stages of growth. The sequence coverages of the control samples are used to identify at least two regions of each genome active in replication. Importance scores and weight scores are assigned to these active regions. The sequence coverages over the active regions obtained for the working sample are used with the importance and weight scores to predict growth and viability of one or more prokaryotic species of the working sample.

### BACKGROUND

The present invention relates to the determination of growth rates and viability from genome coverage, and more specifically, to assessing the growth and hazard potential of one or more species of microorganisms in a multi-species sample.

Metagenomic or metatranscriptomic sequencing datasets allow the determination of the presence or absence of a given organism but do not provide a means of assessing the viability, rate of growth, and/or rate of replication of an organism at the time of sampling. This deficiency can preclude the determination of a hazard potential associated with the organism.

Many methods exist for inferring the different life states of bacteria (Davis, C., “Enumeration of probiotic strains: Review of culture-dependent and alternative techniques to quantify viable bacteria,” Journal of Microbiological Methods, 103, 9-17 (2014); Singer et al., “Capturing the genetic makeup of the active microbiome in situ,” The ISME Journal (2017), 11, 1949-1963). However, no currently implemented method exists for utilizing sequencing data for this purpose.

Most methods of viability determination are based on quantitative PCR (polymerase chain reaction) of pre-rRNA sequences before and after stimulation of the sample with nutrients (Cangelosi et al., “Dead or Alive: Molecular Assessment of Microbial Viability,” Applied and Environmental Microbiology 80, 5884-5891 (2014)). These methods cannot be applied to passively collected data and are tailored to assay only a select set of species, as PCR probes need to be synthesized ahead of time (U.S. Pat. No. 9,115,407 B2). The same probe-related bias persists with methods that are based on fluorescence (e.g., fluorescence activated cell sorting (FACS)).

The advent of high-throughput next-generation sequencing has allowed for sampling the entire microbial community present in a food, environmental, or patient sample. Central to this approach is broad fragmentation of nucleic acids extracted from bacterial cells, referred to herein as “shotgun sequencing.” For each sequencing experiment, researchers have the choice of extracting either RNA or DNA as the primary template of interest. Both can provide insight into the taxonomic and functional characteristics of the microbes, but they skew differently in terms of their coverage of various parts of the investigated genomes and in terms of answering different biological questions.

Computational methods in the bioinformatics field have been developed to align the short reads generated by shotgun sequencing to high quality assembled reference genomes. This is done by minimizing the disagreement in the nucleotides of the two biological sequences being compared.

Alignments of short reads to reference genomes can be seen as a histogram of counts of respective nucleotides along the entire length of the genome template. Commonly, the differential peak heights of this histogram are informative of the sample either having an alternate copy number of a segment compared to the reference or having differential expression for DNA and RNA, respectively (Korem et al., “Growth dynamics of gut microbiota in health and disease inferred from single metagenomic samples,” Science, 2015, 349, 1101-1106).

A general comparison of metagenomic and metatranscriptomic sequence data of a small set of samples validates that some genes are overexpressed compared to their genomic copy number (e.g., ribosomal proteins), suggesting more active replication, while others are underexpressed (e.g., sporulation genes) suggesting senescence (Franzosa et al., “Relating the metatranscriptome and metagenome of the human gut,” Proc. Natl. Acad. Sci. U.S.A., 2014, 111, E2329-38). However, this work has not been used to further strengthen the link between differences in expression and microbial activity, nor has it been used to develop a machine learning method that is capable of predicting replication rates.

In addition to studies in bacteria, some yeast genes are predictive of yeast growth rates measured experimentally (Airoldi et al., “Predicting cellular growth from gene expression signatures,” PLoS Computational Biology, 2009, 5, e1000257). This validates that transcription is altered by the underlying changes in the number of DNA copies. More complex dynamics arise than just a linear amplification of transcript copies, especially in eukaryotes (Bar-Ziv et al., “Dealing with Gene-Dosage Imbalance during S Phase,” Trends in Genetics, 2016, 32(11), 717-723), necessitating a more careful tuning of predictive sequences.

Methods of identifying high growth species in a community sample are needed that do not rely on culturing the samples and are adaptable to machine learning.

### SUMMARY

Accordingly, a method is disclosed, comprising:

sequencing nucleic acids of a non-cultured sample, the sample containing one or more prokaryotic species, thereby producing a plurality of sequences of the nucleic acids, the sequences comprising i) metagenomic sequences corresponding to DNA and ii) metatranscriptomic sequences corresponding to RNA;

mapping the sequences to reference genomes of the one or more prokaryotic species, thereby obtaining i) a list of taxonomically identified prokaryotic species of the sample and ii) coverages of the sequences with respect to two or more regions of the reference genomes active during growth of the prokaryotic species, the regions designated active regions; and

calculating a growth rate and a viability of the one or more prokaryotic species of the sample based on the coverages.

Also disclosed is a system, comprising one or more computer processor circuits configured and arranged to:

sequence nucleic acids of a non-cultured sample, the sample containing one or more prokaryotic species, thereby producing a plurality of sequences of the nucleic acids, the sequences comprising i) metagenomic sequences corresponding to DNA and ii) metatranscriptomic sequences corresponding to RNA;

map the sequences to reference genomes of the one or more prokaryotic species, thereby obtaining i) a list of taxonomically identified prokaryotic species of the sample and ii) coverages of the sequences with respect to two or more regions of the reference genomes active during growth of the prokaryotic species, the regions designated active regions; and

calculate a growth rate and a viability of the one or more prokaryotic species of the sample based on the coverages.

Further disclosed is a computer program product for calculating growth rates and viability of prokaryotic species of a non-cultured sample, the sample comprising one or more prokaryotic species, the computer program product comprising a non-transitory computer readable storage medium having program code embodied therewith, the program code executable by a processing device to perform a method comprising:

sequencing nucleic acids of a non-cultured sample, the sample containing one or more prokaryotic species, thereby producing a plurality of sequences of the nucleic acids, the sequences comprising i) metagenomic sequences corresponding to DNA and ii) metatranscriptomic sequences corresponding to RNA;

mapping the sequences to reference genomes of one or more prokaryotic species, thereby obtaining i) a list of taxonomically identified prokaryotic species of the sample and ii) coverages of the sequences with respect to two or more regions of the reference genomes active during growth of the prokaryotic species, the regions designated active regions; and

calculating the growth rate and the viability of the one or more prokaryotic species of the sample based on the coverages.

The above-described and other features and advantages of the present invention will be appreciated and understood by those skilled in the art from the following detailed description, drawings, and appended claims.

### BRIEF DESCRIPTION OF THE SEVERAL VIEWS OF THE DRAWINGS

FIG. 1, which includes FIGS. 1A-1G, shows a process flow diagram for Part 1 of the disclosed method.

FIG. 2, which includes FIGS. 2A-2H, shows a process flow diagram for Part 2 of the disclosed method.

FIG. 3 is a block diagram of a computer system and computer program code that may be used to implement Parts 1 and 2 of the disclosed method.

FIG. 4 is a flow chart of the process used in the Example.

### DETAILED DESCRIPTION

A method is disclosed for predicting viability and growth rates of at least one prokaryotic species of a working sample (e.g., environmental, food, patient) utilizing nucleic acid (i.e., DNA and/or RNA) sequencing data. The method is not dependent on amplification of the nucleic acid, hybridization of nucleic acid, or physical properties of the nucleic acid once the nucleic acid has been sequenced. The method is general to any prokaryotic organism (i.e., archaea, bacteria), as opposed to a well-characterized single prokaryotic species. The method does not require culturing of the sample or any implicit annotation of the genome of interest. The method is also applicable to samples previously frozen/shipped from a remote location. The method is adaptable to machine learning.

As bacteria reproduce asexually, the increase in their population is accomplished by binary fission, which features among many things the duplication of the circular genome. In general, this occurs via a symmetric process that starts at a known locus, referred to in the art as the origin of replication, and proceeds in both directions along the template genome. This locus has certain identifiable features (e.g., high adenine-thymine content) that promote the replication “machinery” (e.g., DNA polymerase, circular sliding clamp, pentameric clamp loader, helicase, primase, and single-strand binding protein) binding to it.

During the replication process, a transient difference can occur in the copy number of DNA regions close to the origin of replication compared to more distant areas of the genome. The copy number imbalance gathered from a working sample containing multiple bacteria has been found to be useful to infer statistically which species of the working sample are actively replicating and to assign viability scores based on the relative activity of each of the species. The analysis to predict viability is therefore linked to present replication activity rather than to natural selection to regions surrounding the loci (i.e., population evolution).

Other loci of the circular genome can also serve as indicators of active replication. For example, in order to replicate the entire genome and successfully reproduce, bacteria produce more RNA transcripts (and subsequently proteins) related to making the replication machinery and the substrates and energy required therefor. These loci share homology among multiple bacterial genomes, as the need to reproduce and hence replicate one's genome is a universal feature of living organisms. Additionally, bacterial genomes prioritize genes vital for replication and transcription to be clustered closer to the origin of replication (Couturier et al., “Replication-associated gene dosage effects shape the genomes of fast-growing bacteria but only for transcription and translation genes,” Mol. Microbiol., 2006, 59, 1506-1518). The disclosed method utilizes these loci, selecting the most predictive loci in samples containing multiple species.

### Definitions

The following definitions are applicable.

A “contig” is a set of overlapping DNA sequences that together represent a consensus sequence of DNA or a region thereof.

A “consensus sequence” is the calculated order of the most frequent residues found at each position in a sequence alignment.

“Copy number” means the number of copies of a gene or plasmid within a genome. The copy number can vary from individual to individual.

“Coverage” or “depth of coverage” is the number of times a given sequence from a genome is represented in the set of sequences derived from that genome.

“DNA” is deoxyribonucleic acid. DNA can be double-stranded or single-stranded.

A “gene” is the basic unit of heredity, a linear sequence of nucleotides along a segment of DNA that provides the coded instructions for synthesis of RNA, which, when translated into protein, leads to the expression of a hereditary trait.

A “genome” is the total genetic content of a microorganism. In the case of bacteria, the genome is the DNA.

A “ground truth dataset” is a dataset formed by direct observation (measured data) as opposed to data obtained by inference or assumption.

Herein “high-throughput sequencing” (HTS) is any method of sequencing a nucleic acid that is highly parallel and does not involve cloning the nucleic acid. A genome or metagenome is cut into a large number of fragments, and the fragments are sequenced in parallel.

“Homology” refers to the similarity of sequences (e.g., DNA, RNA, Protein, etc.) arising from a common ancestry.

“Hybridization” is the formation of a double-stranded helix from single-stranded complimentary pairs of DNA and/or RNA by annealing.

The term “k-mer” means a sub-sequence of a read obtained through DNA sequencing having k number of nucleotide base units, where k is a positive whole number greater than 1.

Herein, a “database” is an electronic file for storing and retrieving data. Databases are also referred to herein as data tables. Data tables comprise rows and columns (i.e., fields) of data. The rows are formally called tuples or records. A data table comprises one or more records, each record comprising one or more defined fields having respective defined data types (e.g., text, numeric, date, time, memo, and so on) and defined field lengths where applicable. A working data table comprises at least one record containing data in one or more fields of the record. The data tables are located on data storage devices, which can be remote or local relative to the user input/output devices. A “database system” comprises at least one data table and a database management software program for managing the storage and retrieval of data to and from the data tables. The database management programs can be remote or local relative to the data tables and/or the end user. A Relational Database Management System (RDBMS) is a database management system (DBMS) that uses relational techniques for storing and retrieving data using data tables. A relational database system can have many data tables, and each data table can have multiple records and multiple fields within each record. A data table in a relational database system can be accessed using an index. An index is an ordered set of references (e.g., pointers) to the records or rows in a data table. The index is used to access each record in the file using a key (e.g., one or more of the fields of the record or attributes of the row). Without an index, finding information in a large data table would require a resource-intensive time-consuming scan (e.g., linearly) of each record of a table. Indexes provide a faster alternate technique of accessing data contained in one or more data tables that are linked by a common key. Users can create indexes on a table after the table is built. An index is based on one or more columns (fields) of a given table.

A “k-mer database” is a database in which a given record comprises a field for storing a k-mer of a nucleic acid sequence of one or more organisms. Another field of the record stores a taxonomic ID that associates the k-mer to a lowest common ancestor node (LCA) of a taxonomic tree. As will be described below in more detail, other fields of the record can store reference IDs to a reference taxonomy. Still other fields of the record can store metadata associated with the k-mer and/or the nucleic acid sequence from which the k-mer originated.

A “locus” (plural loci) is a position on a genome of a region of interest (e.g., gene, regulatory element, origin of replication).

A “metagenome” is all the genetic information from a microbiome (see microbiome below).

“Metagenomics” is the analysis or study of metagenomes.

“Metatranscriptome” is the collection of all RNA transcripts from a microbiome (see microbiome below)

“Metatranscriptomics” is the analysis or study of metatranscriptomes.

A “microbiome” is a community of microorganisms that inhabit a particular environment (e.g., microbes of the human gut), or a sample taken therefrom.

“Origin of replication” is the locus at which DNA replication begins.

A “plasmid” is a self-replicating extrachromosomal circular DNA that replicates independently of the bacterial chromosome and carries genes for functions not essential for growth.

“RNA” is ribonucleic acid.

“mRNA” refers to messenger RNA. The mRNA codes for amino acid sequences of proteins.

“rRNA” refers to ribosomal RNA.

“tRNA” refers to transfer RNA. A tRNA transports a specific amino acid to a ribosome for synthesis of a protein.

An “RNA transcript” is an RNA produced through the process of transcription of DNA.

“Sample” means any sample capable of undergoing analysis using the disclosed methods. The sample can contain one or more microorganisms. Non-limiting samples include water samples obtained from tap water, lakes, streams, field runoff, and sewage; swabbed samples from contact surfaces (e.g., building surfaces, countertops, furniture, utensils, clinical instruments, computer hardware, cell phones, door handles, doors, windows, screens, cabinets, cabinet doors, sinks, faucet); animal samples (e.g., blood, blood plasma, serum, cells, a cellular extract, a cellular aspirate, expectorant, sputum, saliva, mucous, urine, sweat, tears); and samples obtained from food, food-handling equipment, and surfaces contacted by food. The samples can be a solid or liquid containing water or no water.

“Sequencing” refers to a process of determining the precise order of residues (e.g., nucleotides) in a nucleic acid (e.g., DNA, RNA).

“Sequence” refers to a fragment of a nucleic acid (RNA or DNA) that has been sequenced (i.e., the order of base pairs is known).

A “sequence read” or “read” is a finite length or fragment of a nucleic acid that is output by a sequencing instrument. For example, a read from an Illumina sequencer is 100-150 base pairs in length today. Sequencing may also be done on “paired end” reads where two reads are connected by a spacer (that is not read), increasing the effective read length to 300 or more and covering a larger region of the genome.

A “sequence alignment” is a way of arranging sequences to identify regions of similarity, which may be a consequence of functional, structural, or evolutionary relationships between the sequences.

“Shotgun sequencing” is a quasi-random process in which a nucleic acid is broken up into many random smaller fragments that are individually sequenced. The sequences are ordered based on overlapping regions of genetic code, and reassembled into the complete sequence of the nucleic acid.

“Taxonomy” is a biological scheme of classification of organisms. Herein, for prokaryotes, the heirarchy is domain, kingdom, division, phylum, class, order, family, genus, species, sub-species, and strain.

A “taxonomic tree” herein is a data structure for classifying organisms. The taxonomic tree comprises nodes (i.e., taxa, singular taxon) that are grouped into “parent nodes” linked to “child nodes”. Parent nodes are depicted above child nodes in the tree diagram. Child nodes are taxonomic descendants of parent nodes. For example, a genus (parent node) can be linked to two or more species (child nodes). The taxonomic tree can be rooted (i.e., known ancestral root) or unrooted (i.e., unknown ancestral root), bifurcating (i.e., two child nodes per parent node) or multi-furcating (i.e., more than two child nodes per parent node). Typically, the taxonomic tree is in the form of a “binary tree” (i.e., each parent node has two child nodes). A “leaf node” is a child node having no descendants (e.g., the species of a genus). In the self-consistent taxonomy, each leaf node has one genome. “Internal nodes” are all nodes other than the leaf nodes.

“Transcription” is the process of forming an RNA from a DNA template.

The abbreviation “bp” means “base-pair” (e.g., a read of 100-bp means that one DNA read has 100 nucleotides in the polymer chain.

“Miscalling” refers to a sequencing error where a nucleotide in a sequence read is different from the true nucleotide.

A quality value is an assigned value given to each nucleotide in a sequence read that reflects the likelihood of miscalling the nucleotide. The higher the quality value is, the lower the likelihood of miscalling.

A “reference genome” is a genome from the same species or close species that has already been sequenced.

“Mapping” a sequence read is a process of finding the position or coordinate of a sequence read on the reference genome.

A “perfect match prefix” is a k-mer of a sequence read that is identical to, or a perfect match to some equal-length k-mer(s) of the reference genome. The k-mer of the sequence read is used to initially anchor the sequence read on the reference genome.

Base substitution: After a sequence read is mapped to the reference genome, certain bases are different from the corresponding bases on the reference genome.

Insertion: Compared with the reference genome, some continuous bases are inserted between two adjacent bases on the sequence read.

Deletion: Compared with the reference genome, the sequence read loses some continuous bases.

INDEL: an insertion or deletion in a read when trying to find the best alignment of a read to a reference genome.

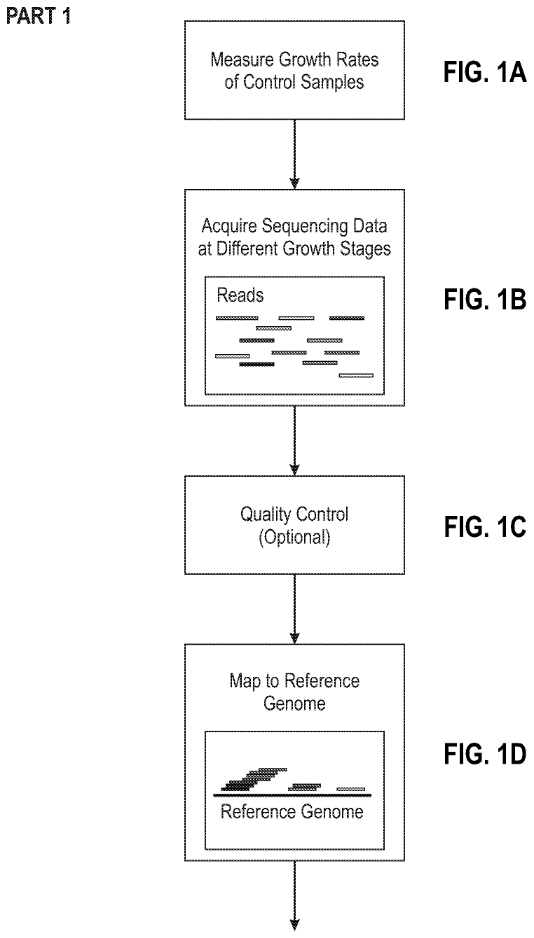

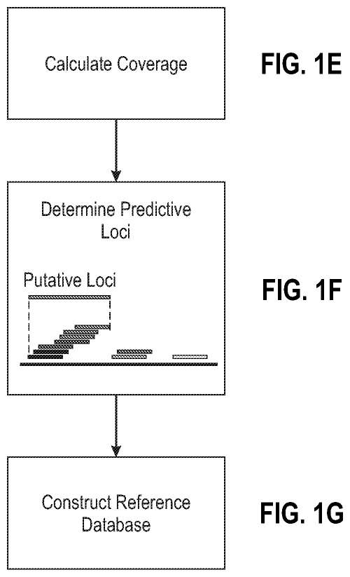

The disclosed method is composed of two parts. Preferably, each part is performed using a computer system. In Part 1, depicted in the process flow diagram of FIG. 1, which includes FIGS. 1A-1G, a reference database is created comprising DNA and/or RNA sequences obtained at different stages of growth of control samples of microorganisms of interest. The sequences obtained at various growth stages are mapped to a known sequenced whole genome of the microorganism. The coverages of the sequences over different regions of the genomes as a function of growth stage are calculated, thereby identifying loci important to replication of the microorganism. The regions of the genome active in replication are referred to herein as “active regions.” The active regions include the origin of replication and any region of the genome actively producing components for replication of the microorganism (i.e., the replication “machinery”). At least two active regions are identified based on the time-dependent coverages.

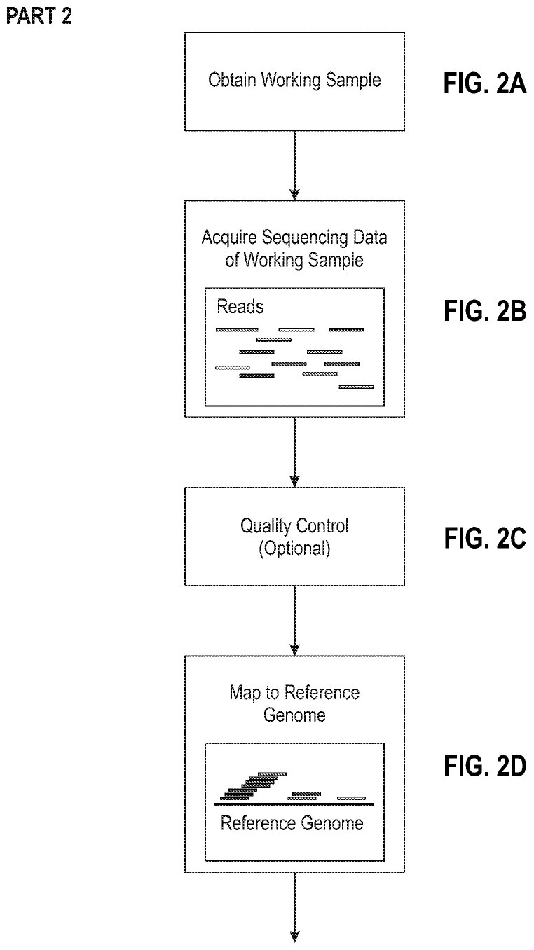

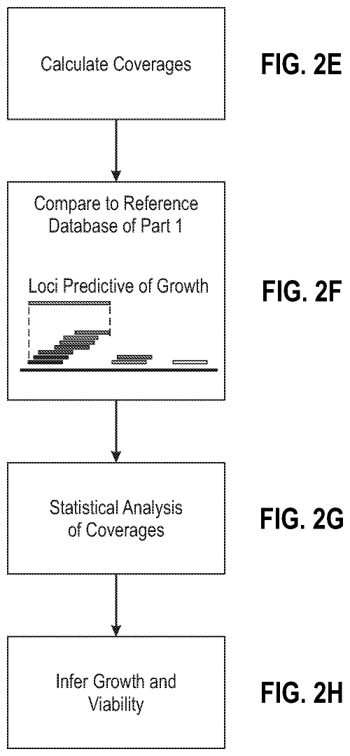

In Part 2, depicted in the process flow diagram of FIG. 2, which includes FIGS. 2A-2H, the DNA and/or RNA of a working sample obtained from the environment, which can contain one or more prokaryotic species, is sequenced and mapped to a collection of known sequenced reference genomes, and the coverages calculated. At this point, the measured observations of the control samples of the reference database are used to perform a statistical analysis of the coverages of the working sample, culminating in a viability score and growth score of one or more of the microorganisms of the working sample.

More specifically, the reference database is used as a component of computer system to perform a statistical analysis of the metagenomic coverage data of the working sample, from which is generated a viability and/or growth score of one or more microorganisms therein. Both viability and growth rate predictions are based on the alignment of sequenced nucleic acid fragments over the active regions defined in the reference database. The computer system includes a computer program and end user interface, described in more detail further below.

Parts 1 and 2 are described in more detail in the following sections.

### Part 1. Creation of the Reference Database

### Measure Growth Rates

To construct a reference database, the population growth rate of one of more bacterial control samples are measured with time (FIG. 1A). Bacterial control samples can be constructed through laboratory grown strains over multiple time periods to observe doubling time.

To create a control sample, the user selects a bacterial isolate and measures an initial quantity or concentration of bacterial cells. Non-limiting methods of measuring bacterial growth rates include i) optical density (OD) measurements, ii) CFU (colony forming units) counting assays, and iii) quantitative PCR. These methods can be used singularly or in combination. The initial quantity of bacterial cells is then added to a liquid growth medium and incubated at a user selected temperature that supports growth (25° C. or 37° C. are most common). A standard bacterial growth assay is performed as follows. Samples of the liquid culture are taken at user-defined times that should include the 3 phases of bacterial growth: the lag phase, log phase, and stationary phase. It should be understood that different bacteria can have different rates of growth that affect the choice of time interval between samples. The bacterial growth of each of the samples is then characterized using one or a combination of the above-described methods of determining the concentration of bacterial cells. The samples are also sequenced (below). As an example, Escherichia coli completes all three growth phases in approximately 20 hours at 37° C. The user may elect to sample the liquid culture once every two hours for preparation for sequencing. After the 20 hours and 11 time points (0-20), the user has samples from each phase of growth.

### Sequencing

At each time interval, the nucleic acids (i.e., RNA and/or DNA) are extracted from the control samples. The extracted nucleic acids are then subjected to high-throughput sequencing (FIG. 1B), resulting in time-dependent raw sequences (reads) of varying nucleotide base length. Non-limiting methods of DNA/RNA high through-put sequencing include massively parallel signature sequencing (or MPSS), Polony sequencing, 454 pyrosequencing method, Illumina (Solexa) sequencing, SOLiD sequencing, ion semiconductor sequencing, DNA nanoball sequencing, heliscope sequencing, single molecule real time sequencing (SMRT sequencing), solid state nanopore sequencing, protein based nanopore sequencing, sequencing by electrical tunneling currents, sequencing by matrix-assisted laser desorption ionization time-of-flight mass spectrometry (MALDI-TOF MS), microfluidic Sanger sequencing, transmission electron microscopy DNA sequencing, RNA polymerase (RNAP) sequencing method, in vitro virus high throughput sequencing (IVV-HiTSeq), and sequencing by hybridization. Multiple, fragmented sequences can be assembled together by software on the basis of their overlapping areas. The foregoing sequencing methods can be used singularly or in combination. Preferably, the sequencing method operates in a parallel mode (characterizing many sequences concurrently).

### Quality Control (QC)

Optionally, quality control of the raw sequences can be performed by purging sequences of poor quality, removing contaminating sequences introduced by the sequence methodology, and/or removing (trimming) sequences of low complexity (FIG. 1C). Sequences of low complexity include those containing contiguous repeating pairs of two nucleotides (e.g., . . . (GA)ₙ . . . where n is a whole number greater than about 3). Contaminating DNA/RNA sequences can originate from inadvertent human, other animal, and/or plant contact with the working sample. Non-limiting algorithms and software programs for trimming and cleanup of raw sequences include SolexaQA DynamicTrim, FASTX-ToolKit, ConDeTri, NGS QC Toolkit, FASTQC, and Trimmamatic. Preferably, quality control is performed by FASTQC and/or Trimmamatic, thereby forming a set of “clean” sequences of the control sample.

Optionally, the raw sequences (or the clean sequences) can be assembled by software to form what is referred to herein as raw contigs or clean contigs, respectively (not shown). If using assembled contigs, the assembly must represent complete coverage of the genome or substantially complete coverage of the genome.

### Mapping to a Reference Genome

Each of the time-dependent sets of raw sequences, clean sequences, raw contigs, and/or clean contigs of the control sample, referred to herein collectively as control reads, are then mapped (aligned) to a known reference genome assembly of the microorganism of the control sample (FIG. 1D). Reference genomes may be chosen by the user and can be from a public or private repository. Public sources can include Genbank, NCBI RefSeq, or others as commonly known by those skilled in the art. Mapping can be performed using short read alignment methods, pair-wise alignment methods, and/or multiple alignment methods. Non-limiting examples of software tools for short read alignment methods include Arioc, BFAST, BLASTN, Bowtie, BWA (Burrows-Wheeler aligner, Li et al., “Fast and accurate short read alignment with Burrows-Wheeler transform,” Bioinformatics, 2009, 25, 1754-1760), CUSHAW, Geneious Assembler, GMAP, and MOSAIK. Non-limiting examples of software tools for pair-wise alignment of sequences include ALLALIGN, BLASTZ, DNASTAR, GAP, GGSEARCH, GLSEARCH, LALIGN, NW-align, SPA, SWIFT, and Wordmatch. Non-limiting examples of software tools for multiple sequence alignment include CHAOS, Clustal W, DNA Baser, DNASTAR, Kalign, MAFFT, MUSCLE, T-Coffee, and UGENE. The mapping can be performed by a program that maps k-mers of the control reads to the known whole reference genome.

### Calculate Coverages

The per-position-coverage and windows of the set of mapped control reads are then calculated (FIG. 1E). A “window” is a region of the genome, the size of which can be set manually or by software. Per-position-coverage is the number of reads corresponding to a mapping location that overlaps a given single nucleotide position.

Optionally, the mapped control reads can be filtered based on a coverage threshold (not shown), effectively removing them.

Optionally, the coverage data of the mapped control reads can be smoothed using LOESS (locally weighted polynomial regression) or another genome feature-aware method to remove unlikely spikes of coverage (not shown). More specifically, LOESS is a method for capturing diverse score-length relationships in sequence data and correcting sequence scores for length-dependent artifacts.

### Identify Active Regions

The time-dependent coverages of the mapped control reads are then analyzed in order to identify all “active regions” of the genomes that are associated with growth and viability of the microorganism of the control sample (FIG. 1F). The analysis can be accomplished in a number of ways, ranging from traditional differential expression methods implemented in well-known packages such as DESeq2 or limma+voom to more involved approaches using sequence kernels and SVM/SVR or tree-based methods (random forest and gradient boosting). The key outputs of this step, disregarding the specific implementation chosen, are (a) an importance score for each of the active regions and (b) a weight score for each of the active regions (or similar transformation method for applying the learned predictors to a working sample).

To evaluate the performance of different methods at predicting replication rate, standard evaluation criteria can be used such as, for example, mean squared error or mean absolute error. To assess performance at predicting viability using the ground truth data generated from the control experiment and comparing to the predicted features from a model trained on that data, standard metrics can be used such as misclassification rate, sensitivity, specificity, and other techniques known in the art.

As used herein, the term “specificity” refers to a measure of correctly predicting growth rate and viability by identifying genomic regions (based on coverages) that should not be included in the estimation of growth and viability (i.e., a measure of true negatives). The term “sensitivity” refers to a measure of correctly predicting growth rate and viability by identifying genomic regions (based on coverages) that should be included in the estimation of growth and viability (i.e., a measure of true positives).

### Create Reference Database

The reference database is then constructed as a set of sequences (FIG. 1G), which comprises the predictive sequences as determined from the time-dependent sequence data obtained at different stages of growth, the identified active regions of each genome during growth, the importance scores of the active regions, and weight scores for the active regions. The reference database can comprise the time-dependent sequence data of one or more microorganisms, more specifically tens, hundreds, or thousands of microorganisms. The time-dependent sequence data of the reference database can be in the form of k-mers of the control reads.

The reference database is used in Part 2 to predict growth rates of one or more microorganism of a working sample.

### Part 2. Viability and Growth Rate Prediction of a Working Sample

### Collect Working Sample

In Part 2 of the method, a working sample is collected (FIG. 2A) containing one or more microorganisms. The working sample can be gathered under natural conditions, without bias toward the species present. As non-limiting examples, samples can be drawn from animal fluids (e.g., blood, urine), lakes, streams, and foods.

### Sequencing Working Sample

Nucleic acids (i.e., RNA and/or DNA) are extracted from the working sample without prior nutrient stimulation of the working sample or use of other DNA amplification techniques. The extracted nucleic acids are then subjected to high throughput sequencing (FIG. 2B) as described above, producing raw working sequences.

### Quality Control

Optionally, quality control of the raw working sequences can be performed as described above by removing sequence data of poor quality introduced by the sequencing methodology, removing contaminating sequences, and/or removing low complexity sequences (FIG. 2C). The result is a set of clean working sequences of the metagenome.

Optionally, the raw working sequences or clean working sequences are assembled to form raw working contigs or clean working contigs, respectively (not shown).

### Mapping

The raw working sequences, clean working sequences, raw working contigs, or clean working contigs, collectively referred to herein as working reads, can then be mapped to the collection of known taxonomically classified whole reference genomes of prokaryotes (FIG. 2D) of the above-described reference database created in Part 1 or a different reference database (e.g., PATRIC database). The mapping allows identification of one or more species of microorganisms of the working sample. The set of whole reference genomes can be in the form of k-mers, and the mapping can be performed by a program that maps k-mers of the working reads to the set of k-mers of the whole reference genomes.

### Coverages

The mapping also determines the per-position-coverage (FIG. 2E) and windows of each of the working reads over the active regions linked to viability and growth identified in Part 1. Optionally, coverage thresholds over the active regions can be used to filter working reads based on the coverage threshold (not shown). Optionally, the sequence coverage data can be smoothed (not shown). For example, smoothing using a generic method of non-parametric regression (e.g., LOESS) can be performed to remove coverage spikes arising from technical biases. A more specialized smoothing method that takes into account specific genome characteristics can also be used if that information is available.

In the next step (FIG. 2F), the computer program and/or an end-user selects the coverages of working reads that correspond to the active regions identified in the time-dependent growth data of the reference database formed in Part 1 above (see FIG. 1F). That is, the computer program and/or end-user defines a set of features that correspond to the reference database regions, coverages, and associated scores which include the relevant coverages combined with learned weights from the reference database to perform a statistical analysis on the coverages (FIG. 2G) and calculate a predicted growth rate and viability of one or more of the prokaryote species of the working sample (FIG. 2H). The calculation can be performed using one method or a weighted combination of different methods.

The reference database formed in Part 1 can be further refined using the positive and/or negative outcomes of the predicted growth rates and viability scores in order to improve the likelihood and accuracy of future successful predictions.

### Microorganisms

Microorganisms include bacteria, fungi, viruses, protozoans, and parasites.

Exemplary non-limiting bacterial species include Acetobacter aurantius, Acinetobacter baumannii, Actinomyces israelii, Agrobacterium radiobacter, Agrobacterium tumefaciens, Akkermansia muciniphila, Alistipes_onderdonkii, Alistipes putredinis, Anaplasma phagocytophilum, Azorhizobium caulinodans, Azotobacter vinelandii, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus Thuringiensis, Bacteroides fragilis, Bacteroides gingivalis, Bacteroides melaninogenicus (also known as Prevotella melaninogenica), Bartonella henselae, Bartonella quintana, Bordetella, Bordetella bronchiseptica, Bordetella pertussis, Borrelia afzelii, Borrelia burgdorferi, Borrelia garinii, Borrelia recurrentis, Brucella abortus, Brucella canis, Brucella melitensis, Brucella suis, Burkholderia mallei, Burkholderia pseudomallei, Burkholderia cepacia, Calymmatobacterium granulomatis, Campylobacter, Campylobacter coli, Campylobacter fetus, Campylobacter jejuni, Campylobacter pylori, Chlamydophila pneumoniae (previously called Chlamydia pneumoniae), Chlamydophila psittaci (previously called Chlamydia psittaci), Chlamydia trachomatis, Clostridium botulinum, Clostridium difficile, Clostridium perfringens (previously called Clostridium welchii), Clostridium tetani, Corynebacterium diphtheriae, Corynebacterium fusiforme, Coxiella burnetii, Ehrlichia canis, Ehrlichia chaffeensis, Enterobacter cloacae, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactococcus lactis, Legionella pneumophila, Leptospira interrogans, Leptospira santarosai, Leptospira weilii, Leptospira noguchii, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycobacterium ulcerans, Mycoplasma fermentans, Mycoplasma genitalium, Mycoplasma hominis, Mycoplasma penetrans, Mycoplasma pneumoniae, Neisseria gonorrhoeae, Neisseria meningitidis, Pasteurella multocida, Pasteurella tularensis, Pepto streptococcus, Porphyromonas gingivalis, Prevotella melaninogenica (previously called Bacteroides melaninogenicus), Pseudomonas aeruginosa, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Shigella sonnei, Spirillum volutans, Streptococcus agalactiae, Staphylococcus aureus, Staphylococcus epidermidis, Staphylococcus saprophyticus, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Streptococcus viridans, Treponema pallidum, Treponema denticola, Ureaplasma urealyticum, Vibrio cholerae, Vibrio comma, Vibrio parahaemolyticus, Vibrio vulnificus, Yersinia enterocolitica, Yersinia pestis, Yersinia pseudotuberculosis,

Non-limiting exemplary viruses include the family Retroviridae, such as human deficiency viruses, such as HIV-I (also referred to as HTLV-III), HIV-II, LAC, IDLV-III/LAV, HIV-III or other isolates such as HIV-LP, the family Picornaviridae, such as poliovirus, hepatitis A, enteroviruses, human Coxsackie viruses, rhinoviruses, echoviruses, the family Calciviridae, such as viruses that cause gastroenteritis, the family Togaviridae, such as equine encephalitis viruses and rubella viruses, the family Flaviviridae, such as dengue viruses, encephalitis viruses and yellow fever viruses, the family Coronaviridae, such as coronaviruses, the family Rhabdoviridae, such as vesicular stomata viruses and rabies viruses, the family Filoviridae, such as Ebola viruses, the family Paramyxoviridae, such as parainfluenza viruses, mumps viruses, measles virus and respiratory syncytial virus, the family Orthomyxoviridae, such as influenza viruses, the family Bungaviridae, such as Hataan viruses, bunga viruses, phleoboviruses and Nairo viruses, the family Arena viridae, such as hemorrhagic fever viruses, the family Reoviridae, such as reoviruses, orbiviruses and rotaviruses, the family Bimaviridae, the family Hepadnaviridae, such as hepatitis B virus, the family Parvoviridae, such as parvoviruses, the Papovaviridae, such as papilloma viruses and polyoma viruses, the family Adenoviridae, such as adenoviruses, the family Herpesviridae, such as herpes simplex virus (HSV) I and II, varicella zoster virus and pox viruses, or the family Iridoviridae, such as African swine fever virus). The virus can be an unclassified virus, such as the etiologic agents of Spongiform encephalopathies, the agent of delta hepatitis, the agents of non-A, non-B hepatitis (class 1 enterally transmitted; class 2 parenterally transmitted such as Hepatitis C); Norwalk and related viruses and astroviruses.

Other non-limiting exemplary viruses include Varicella-zoster virus, Epstein-barr virus, Human cytomegalovirus, Human herpesvirus type 8, Human papillomavirus, BK virus, JC virus, Smallpox, Parvovirus B19, poliovirus, yellow fever virus, West Nile virus, TBE virus, Rubella virus, Hepatitis E virus, Influenza virus, Lassa virus, Crimean-Congo hemorrhagic fever virus, Hantaan virus, Marburg virus, Coltivirus, Banna virus, and zika virus.

Non-limiting exemplary fungi include Candida albicans, Aspergillus fumigatus, Aspergillus flavus, Aspergillus clavatus, Cryptococcus neoformans, Cryptococcus laurentii, Cryptococcus albidus, Cryptococcus gattii, Histoplasma capsulatum, Pneumocystis jirovecii, Pneumocystis carinii, and Stachybotrys chartarurn.

Non-limiting exemplary protozoa include Entamoeba histolytica, Entamoeba coli, Entamoeba dispar, Entamoeba moshkovskii, Entamoeba Bangladeshi, Entamoeba hartmanni, Dientamoeba fragilis, Endolimax nana, Lodarnoeba butschlii, Plasmodium malariae, Plasmodium falciparum, Plasmodium vivax, Plasmodium ovale, Naegleria fowleri, Acanthamoeba species, Balamuthia mandrillaris, Sappinia diploidea, Giardia larnblia, Giardia intestinalis, Giardia duodenalis, Toxoplasma gondii, Nippostrongylus brasiliensis, Cryptosporidium parvum, Cryptosporidium hominis, Cryptosporidium cams, Cryptosporidium felis, Cryptosporidium meleagridis, Cryptosporidium muris, Trichomonas vaginalis, Trypanosoma cruzi, Leishmania major, Leishmania tropica, Leishmania barziliensis, Leishmania mexicana, Leishmania guyanesis, Leishmania panamensis, and Trypanosoma brucei.

### Computer Hardware and Software

The computer system for implementing the present invention can take the form of an entirely hardware embodiment, an entirely software embodiment (including firmware, resident software, microcode, etc.), or a combination of software and hardware that may all generally be referred to herein as a “circuit,” “module,” or “system.”

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

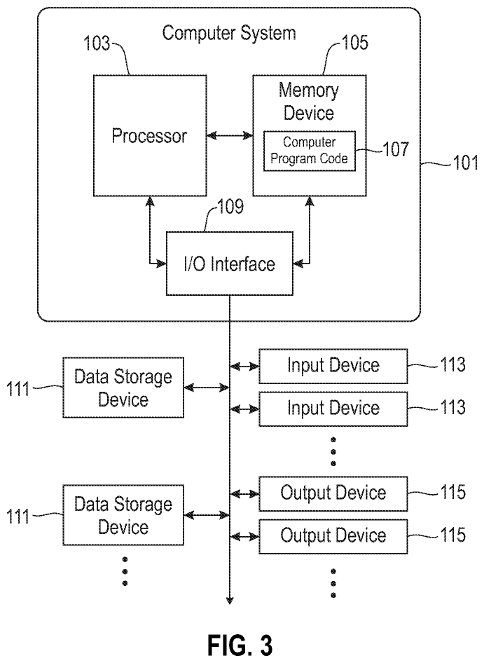

FIG. 3 is a block diagram of a computer system and computer program code that may be used to implement a method of processing, including natural-language processing, to generate a reference database of Part 1 of the disclosed method and predict growth and viability of microorganisms of a working sample of Part 2 of the method. The computer system and program code can also be used to implement a method of processing, including natural-language processing, utilizing the reference database of Part 1 to conduct taxonomic profiling of working samples containing one or more microorganisms.

In FIG. 3, computer system 101 comprises a processor 103 coupled through one or more I/O Interfaces 109 to one or more hardware data storage devices 111 and one or more I/O devices 113 and 115. Hardware data storage devices 111 can contain the reference k-mer database and/or the self-consistent k-mer database.

Hardware data storage devices 111 may include, but are not limited to, magnetic tape drives, fixed or removable hard disks, optical discs, storage-equipped mobile devices, and solid-state random-access or read-only storage devices. I/O devices may comprise, but are not limited to: input devices 113, such as keyboards, scanners, handheld telecommunications devices, touch-sensitive displays, tablets, biometric readers, joysticks, trackballs, or computer mice; and output devices 115, which may comprise, but are not limited to printers, plotters, tablets, mobile telephones, displays, or sound-producing devices. Data storage devices 111, input devices 113, and output devices 115 may be located either locally or at remote sites from which they are connected to I/O Interface 109 through a network interface.

Processor 103 may also be connected to one or more memory devices 105, which may include, but are not limited to, Dynamic RAM (DRAM), Static RAM (SRAM), Programmable Read-Only Memory (PROM), Field-Programmable Gate Arrays (FPGA), Secure Digital memory cards, SIM cards, or other types of memory devices.

At least one memory device 105 contains stored computer program code 107, which is a computer program that comprises computer-executable instructions. The stored computer program code can include a program for natural-language processing that implements the disclosed methods. The data storage devices 111 may store the computer program code 107. Computer program code 107 stored in the storage devices 111 can be configured to be executed by processor 103 via the memory devices 105. Processor 103 can execute the stored computer program code 107.

Thus the present invention discloses a process for supporting computer infrastructure, integrating, hosting, maintaining, and deploying computer-readable code into the computer system 101, wherein the code in combination with the computer system 101 is capable of performing the analysis of sequence data pertinent to the formation of the self-consistent k-mer database from a reference k-mer database, and generating reports therefrom. The computer system 101 is capable of performing the analysis of sequence data of a sample pertinent to the determination of identifying species using the self-consistent k-mer database.

Any of the components of the present invention could be created, integrated, hosted, maintained, deployed, managed, serviced, supported, etc. by a service provider. Thus, the present invention discloses a process for deploying or integrating computing infrastructure, comprising integrating computer-readable code into the computer system 101, wherein the code in combination with the computer system 101 is capable of performing the analysis of sequence data pertinent to the determination of identifying the viable species of the sample.

One or more data storage units 111 (or one or more additional memory devices not shown in FIG. 3) may be used as a computer-readable hardware storage device having a computer-readable program embodied therein and/or having other data stored therein, wherein the computer-readable program comprises stored computer program code 107. Generally, a computer program product (or, alternatively, an article of manufacture) of computer system 101 may comprise said computer-readable hardware storage device.

While it is understood that program code 107 may be deployed by manually loading the program code 107 directly into client, server, and proxy computers (not shown) by loading the program code 107 into a computer-readable storage medium (e.g., computer data storage device 111), program code 107 may also be automatically or semi-automatically deployed into computer system 101 by sending program code 107 to a central server (e.g., computer system 101) or to a group of central servers. Program code 107 may then be downloaded into client computers (not shown) that will execute program code 107.

Alternatively, program code 107 may be sent directly to the client computer via e-mail. Program code 107 may then either be detached to a directory on the client computer or loaded into a directory on the client computer by an e-mail option that selects a program that detaches program code 107 into the directory.

Another alternative is to send program code 107 directly to a directory on the client computer hard drive. If proxy servers are configured, the process selects the proxy server code, determines on which computers the proxy servers' code should be placed, transmits the proxy server code, and then installs the proxy server code on the proxy computer. Program code 107 is then transmitted to the proxy server and stored on the proxy server.

In one embodiment, program code 107 is integrated into a client, server and network environment by providing for program code 107 to coexist with software applications (not shown), operating systems (not shown) and network operating systems software (not shown) and then installing program code 107 on the clients and servers in the environment where program code 107 will function.

The first step of the aforementioned integration of code included in program code 107 is to identify any software, including the network operating system (not shown), on the clients and servers where program code 107 will be deployed, that is required by program code 107 or that works in conjunction with program code 107. This identified software includes the network operating system, where the network operating system comprises software that enhances a basic operating system by adding networking features. Next, the software applications and version numbers are identified and compared to a list of software applications and correct version numbers that have been tested to work with program code 107. A software application that is missing or that does not match a correct version number is upgraded to the correct version.

A program instruction that passes parameters from program code 107 to a software application is checked to ensure that the instruction's parameter list matches a parameter list required by the program code 107. Conversely, a parameter passed by the software application to program code 107 is checked to ensure that the parameter matches a parameter required by program code 107. The client and server operating systems, including the network operating systems, are identified and compared to a list of operating systems, version numbers, and network software programs that have been tested to work with program code 107. An operating system, version number, or network software program that does not match an entry of the list of tested operating systems and version numbers is upgraded to the listed level on the client computers and upgraded to the listed level on the server computers.

After ensuring that the software, where program code 107 is to be deployed, is at a correct version level that has been tested to work with program code 107, the integration is completed by installing program code 107 on the clients and servers.

Embodiments of the present invention may be implemented as a method performed by a processor of a computer system, as a computer program product, as a computer system, or as a processor-performed process or service for supporting computer infrastructure.

### EXAMPLE

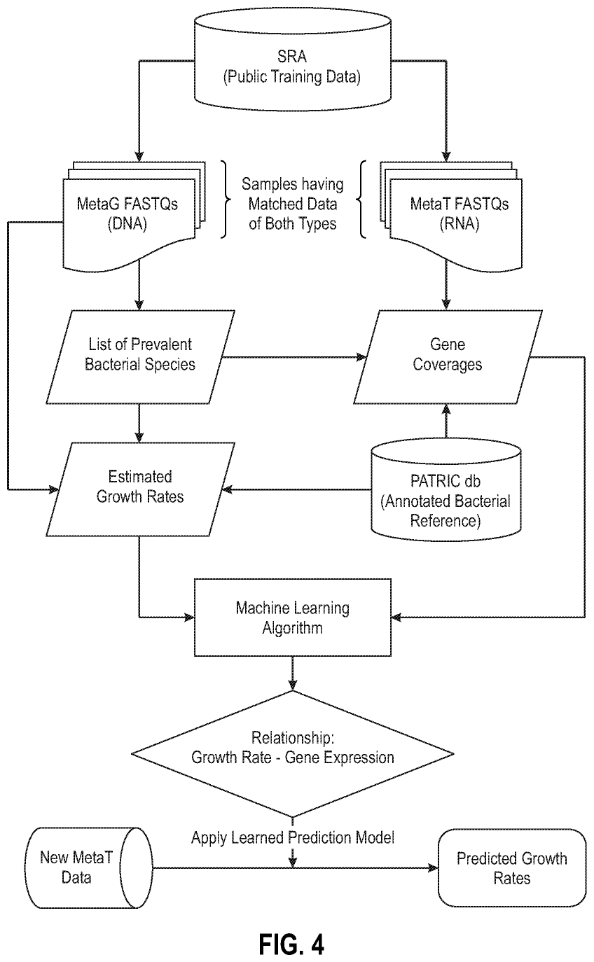

The following example illustrates the practice of the invention and is depicted in the flow chart of FIG. 4. For this example, a previously published set of DNA and RNA sequence data containing matched metagenomes and metatranscriptomes was used as a working sample. This previously generated sequence dataset is available under NCBI BioProject, ID PRJNA289586 in the Sequence Read Archive (SRA) and is treated herein as the sequence data of separate working samples generated from multiple samples of human gut fluids taken from patients with type I diabetes to generate one model (set of reference database entries).

The raw nucleotide sequences (reads) were downloaded via NCBI's prefetch and Fastq-dump utilities. Quality control produced minimally processed paired-end files with technical reads (i.e., identical reads) removed and left- and right-clipping performed.

The metagenomic subset of the working reads was input to a taxonomic profiling tool (Kraken), which generated a list of prevalent bacterial species most likely to be in the sample. Using this list, the metagenomic reads were aligned to representative reference genomes of each of the bacterial species. The resulting alignments were stored in sequence alignment map (SAM) files.

The SAM file was then assessed with the iRep software program found in the public Github repository [www.github.com/christophertbrown/iRep], which generated an estimated growth rate for each species of the working sample. iRep implements the method of Brown et al., “Measurement of bacterial replication rates in microbial communities”, Nature Biotechnology 2016, pp 1256-1263). The growth rate estimates were collated into a single data file with the following columns: sample, species, and growth. Table 1 illustrates a small portion of this data file for three bacteria, Akkermansia_muciniphila, Alistipes_onderdonki, and Alistipes putredinis.

TABLE 1

| | | |
|---|---|---|
| | | Estimated Growth |
| Sample | Species | From iRep |

| | | |
|---|---|---|
| M01.1-V1-stool-metaG | Akkermansia_muciniphila_55290 | 11.23080943 |
| M01.1-V1-stool-metaT | Akkermansia_muciniphila_55290 | 27.01000027 |
| M01.1-V2-stool-metaG | Akkermansia_muciniphila_55290 | 12.14307422 |
| M01.1-V2-stool-metaT | Akkermansia_muciniphila_55290 | 23.13787288 |
| M01.1-V3-stool-metaG | Akkermansia_muciniphila_55290 | 11.11838087 |
| M01.1-V3-stool-metaT | Akkermansia_muciniphila_55290 | 25.4733627 |
| M01.2-V1-stool-metaG | Akkermansia_muciniphila_55290 | 11.88900562 |
| M01.2-V1-stool-metaT | Akkermansia_muciniphila_55290 | 12.1609952 |
| M01.3-V1-stool-metaG | Alistipes_onderdonkii_55464 | 7.239213301 |
| M01.3-V1-stool-metaT | Alistipes_onderdonkii_55464 | 13.12596331 |
| M01.3-V2-stool-metaG | Alistipes_onderdonkii_55464 | 11.69327166 |
| M01.3-V2-stool-metaT | Alistipes_onderdonkii_55464 | 14.06327732 |
| M01.3-V3-stool-metaG | Alistipes_onderdonkii_55464 | 13.15008164 |
| M01.3-V3-stool-metaT | Alistipes_onderdonkii_55464 | 12.83432479 |
| M01.4-V2-stool-metaG | Alistipes_onderdonkii_55464 | 9.645775763 |
| M01.4-V2-stool-metaT | Alistipes_onderdonkii_55464 | 13.11200164 |
| M01.4-V3-stool-metaG | Alistipes_onderdonkii_55464 | 8.712914127 |
| M01.4-V3-stool-metaT | Alistipes_onderdonkii_55464 | 14.91516554 |
| M01.1-V1-stool-metaG | Alistipes_putredinis_61533 | 2.685606663 |
| M01.1-V1-stool-metaT | Alistipes_putredinis_61533 | 15.43725337 |
| M01.1-V2-stool-metaG | Alistipes_putredinis_61533 | 2.773252374 |
| M01.1-V2-stool-metaT | Alistipes_putredinis_61533 | 12.90564292 |
| M01.1-V3-stool-metaG | Alistipes_putredinis_61533 | 2.733368688 |
| M01.1-V3-stool-metaT | Alistipes_putredinis_61533 | 15.78367135 |
| M01.2-V1-stool-metaG | Alistipes_putredinis_61533 | 3.034251937 |
| M01.2-V1-stool-metaT | Alistipes_putredinis_61533 | 13.27962701 |
| M01.2-V2-stool-metaG | Alistipes_putredinis_61533 | 2.728717809 |

The metatranscriptomic data was aligned to annotated reference genomes of the PATRIC database using the same list of prevalent bacterial species as before, which generated a list of expression levels of the genes for each of the identified bacterial species. PATRIC is an all-bacterial bioinformatics database. Wattam et al., “Improvements to PATRIC, the all-bacterial Bioinformatics Database and Analysis Resource Center,” Nucleic Acids Res., 2017, 45(D1):D535-D542.

The relationship between a real-valued phenotype (growth rate from metagenomic data) and the expression levels of a large selection of genes (median coverages from metatranscriptomic data) was evaluated using a differential expression testing framework called limma with the voom extension, developed by Ritchie et al., “limma powers differential expression analyses for RNA-sequencing and microarray studies,” Nucleic Acids Research, 2015. This generated a collection of species-specific genes that had non-random linear association with estimated growth rate, which was used to predict growth rate of each of the species. Table 2 illustrates a small portion of this data for some of the genes of the species Akkermansia muciniphila. “Log FC” is the estimate of the log2-fold-change corresponding to the effect or contrast between two or more experimental conditions, “Ave_expr” is the average log2-expression level for that gene across all the arrays and channels in the experiment, “t” is the moderated t-statistic, “P Value” is the raw p-value. “Adj.P.Value” is the p-value adjusted for multiple testing. “B” is the log-odds that the gene is differentially expressed. The probability of a gene being differentially expressed increases with increasing B value.

TABLE 2

| Species | Gene | LogFC | Ave_expr | t | P Value | Adj. P. Val | B |
|---|---|---|---|---|---|---|---|
| Akkermansia_muciniphila_55290 | 349741.6.peg.127 | 0.773595 | 3.430266035 | 15.45307905 | 1.58E−10 | 4.61E−07 | 14.11072 |
| Akkermansia_muciniphila_55290 | 349741.6.peg.1945 | 0.747995 | 3.454882607 | 14.10735157 | 5.61E−10 | 8.17E−07 | 12.9926 |
| Akkermansia_muciniphila_55290 | 349741.6.peg.2478 | −0.90841 | 6.758976673 | −10.56062053 | 2.81E−08 | 2.72E−05 | 9.223822 |
| Akkermansia_muciniphila_55290 | 349741.6.peg.128 | 0.599895 | 2.769554071 | 9.369782619 | 1.32E−07 | 9.64E−05 | 7.899176 |
| Akkermansia_muciniphila_55290 | 349741.6.peg.733 | 0.686932 | 2.949909624 | 8.949030099 | 2.37E−07 | 1.38E−04 | 7.354775 |
| Akkermansia_muciniphila_55290 | 349741.6.peg.1163 | −0.83218 | 6.251494199 | −8.090148102 | 8.30E−07 | 3.64E−04 | 6.112148 |
| Akkermansia_muciniphila_55290 | 349741.6.peg.1597 | −0.38101 | 9.887909294 | −8.037950625 | 8.99E−07 | 3.64E−04 | 5.999127 |
| Akkermansia_muciniphila_55290 | 349741.6.peg.596 | −0.43834 | 7.117856912 | −7.96845727 | 9.99E−07 | 3.64E−04 | 5.961572 |
| Akkermansia_muciniphila_55290 | 349741.6.peg.487 | 0.456702 | 4.893687936 | 7.748166092 | 1.40E−06 | 4.01E−04 | 5.631832 |
| Akkermansia_muciniphila_55290 | 349741.6.peg.1467 | 0.535452 | 2.596488529 | 7.698554124 | 1.51E−06 | 4.01E−04 | 5.559853 |
| Akkermansia_muciniphila_55290 | 239935.4.peg.2160 | −0.42715 | 8.900514614 | −7.698075543 | 1.52E−06 | 4.01E−04 | 5.517011 |

With a larger training dataset, and more elaborate machine learning algorithms, predictive performance could by further refined, with the ultimate goal of using only metatranscriptomic data to accurately predict the growth rate that previously would need metagenomic data to estimate.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. When a range is used to express a possible value using two numerical limits X and Y (e.g., a concentration of X ppm to Y ppm), unless otherwise stated the value can be X, Y, or any number between X and Y.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and their practical application, and to enable others of ordinary skill in the art to understand the invention.

What is claimed is:

1. A method, comprising:
sequencing nucleic acids of a non-cultured sample, the sample containing one or more prokaryotic species, thereby producing a plurality of sequences of the nucleic acids, the sequences comprising i) metagenomic sequences corresponding to DNA and ii) metatranscriptomic sequences corresponding to RNA;
mapping the sequences to reference genomes of the one or more prokaryotic species, thereby obtaining i) a list of taxonomically identified prokaryotic species of the sample and ii) coverages of the sequences with respect to two or more regions of the reference genomes active during growth of the prokaryotic species, the regions designated active regions; and
calculating a growth rate and a viability of the one or more prokaryotic species of the sample based on the coverages.

2. The method of claim 1, wherein the sample is selected from the group consisting of environmental samples, medical samples, and food samples.

3. The method of claim 1, wherein the reference genomes of the prokaryotic species are contained in a reference database.

4. The method of claim 3, wherein the reference database comprises i) sequences obtained at different stages of growth of the prokaryotic species, ii) importance scores for the active regions, and iii) weight scores for the active regions, wherein the importance scores and weight scores are used in the calculation of growth rate and viability of the one or more prokaryotic species.

5. The method of claim 1, wherein the method is implemented by a computer system.

6. The method of claim 1, wherein said sequencing is high throughput sequencing.

7. The method of claim 1, wherein the coverages are smoothed using locally weighted polynomial regression.

8. The method of claim 7, wherein the coverages are coverages of the contigs.

9. The method of claim 1, wherein the sequences are in the form of contigs.

10. The method of claim 1, wherein the mapped sequences are filtered based on a coverage threshold over the active regions.

11. The method of claim 1, wherein said mapping is performed using k-mers of the sequences.

12. The method of claim 1, wherein the non-cultured sample is frozen prior to sequencing.

13. A system comprising one or more computer processor circuits configured and arranged to:
sequence nucleic acids of a non-cultured sample, the sample containing one or more prokaryotic species, thereby producing a plurality of sequences of the nucleic acids, the sequences comprising i) metagenomic sequences corresponding to DNA and ii) metatranscriptomic sequences corresponding to RNA;
map the sequences to reference genomes of the one or more prokaryotic species, thereby obtaining i) a list of taxonomically identified prokaryotic species of the sample and ii) coverages of the sequences with respect to two or more regions of the reference genomes active during growth of the prokaryotic species, the regions designated active regions; and
calculate a growth rate and a viability of the one or more prokaryotic species of the sample based on the coverages.

14. The system of claim 13, wherein the system comprises a reference database, the reference database comprising i) whole genomes of the one or more prokaryotic species, ii) importance scores for the active regions of whole genomes, and iii) weight scores for the active regions of the whole genomes, wherein the importance scores and the weight scores are used to calculate the growth rate and the viability of the one or more prokaryotic species.

15. The system of claim 14, wherein the reference database comprises sequences of nucleic acids obtained from control samples of the one or more prokaryotic species at different stages of growth.

16. The system of claim 14, wherein the importance score and the weight score are derived from coverages of the sequences from the control samples, the coverages with respect to the active regions of the whole genomes of the one or more prokaryotic species.

17. A computer program product for calculating growth rates and viability of prokaryotic species of a non-cultured sample, the sample comprising one or more prokaryotic species, the computer program product comprising a non-transitory computer readable storage medium having program code embodied therewith, the program code executable by a processing device to perform a method comprising:
sequencing nucleic acids of a non-cultured sample, the sample containing one or more prokaryotic species, thereby producing a plurality of sequences of the nucleic acids, the sequences comprising i) metagenomic sequences corresponding to DNA and ii) metatranscriptomic sequences corresponding to RNA;
mapping the sequences to reference genomes of the one or more prokaryotic species, thereby obtaining i) a list of taxonomically identified prokaryotic species of the sample and ii) coverages of the sequences with respect to two or more regions of the reference genomes active during growth of the prokaryotic species, the regions designated active regions; and
calculating the growth rate and the viability of the one or more prokaryotic species of the sample based on the coverages.

18. The computer program product of claim 17, wherein said mapping is performed by a method selected from the group consisting of short read alignment methods, pair-wise alignment method, multiple alignment methods, and combinations thereof.

19. The computer program product of claim 17, wherein the reference genomes of the one or more prokaryotic species are contained in a reference database, and the computer program product queries the reference database to perform said mapping.

20. The computer program product of claim 19, wherein the reference database comprises importance scores and weight scores for the active regions of the reference genomes, and the computer program product uses the importance scores and the weight scores to perform said calculating of the growth rate and the viability of the one or more prokaryotic species.
